(19) United States
(12) Patent Application Publication
(10) Pub. No.: US 2013/0191940 A1
(43) Pub. Date: Jul. 25, 2013

(54) DOMINANT EARLINESS MUTATION AND GENE IN SUNFLOWER (HELIANTHUS ANNUUS)

(71) Applicant: DOW AGROSCIENCES LLC, Indianapolis, IN (US)

(72) Inventors: Todd James Gerdes, Breckenridge, MN (US); Mark Christopher, Spring Valley, WI (US); Robert Benson, Ellsworth, WI (US); Wenxiang Gao, Zionsville, IN (US)

(73) Assignee: DOW AGROSCIENCES LLC, Indianapolis, IN (US)

(21) Appl. No.: 13/840,372

(22) Filed: Mar. 15, 2013

Related U.S. Application Data
(62) Division of application No. 12/866,819, filed on Nov. 15, 2010.
(60) Provisional application No. 61/028,052, filed on Feb. 12, 2008.

(51) Int. Cl. A01H 5/02

(52) U.S. Cl. CPC A01H 5/02
USPC 800/267; 800/322; 435/6.11

(57) ABSTRACT

The subject invention relates in part to the discovery of a spontaneous sunflower mutation. The subject invention involves an “early” mutation and related inbred/hybrid development. The subject invention further provides a single dominant gene that confers earliness in sunflower inbred isolines and near isogenic hybrids. There is no known prior teaching or suggestion of this gene's utility for hybrid development in the industry. The subject invention also provides a new and distinctive sunflower inbred line designated H120R. The invention includes seeds that possess this mutated gene, plants produced by growing these seeds, and progeny thereof that possess this mutated gene and the associated earliness trait. The subject invention also includes methods for producing such sunflower seeds and plants, including inbreds and hybrids. Such plants can be produced by, for example, crossing such an inbred line with itself or with another sunflower line.

### CROSS-REFERENCE TO RELATED APPLICATION

This application is a Divisional of U.S. application Ser. No. 12/866,819, filed Nov. 15, 2010, which is a National Stage filing of International Application Serial No. PCT/US2009/033955, filed Feb. 12, 2009, which claims priority to U.S. Provisional Application No. 61/028,052, filed Feb. 12, 2008, all of which are expressly incorporated herein by reference.

### BACKGROUND OF THE INVENTION

Sunflowers are an important and valuable field crop to supply food for both animals and humans. A continuing goal of plant breeders is to develop stable, high yielding sunflower hybrids that are agronomically sound so that the amount of seed produced on the land used is maximized. To accomplish this goal, the sunflower breeder must select and develop sunflower plants that have the traits that result in superior parental lines for producing hybrids.

Sunflower (Helianthus annuus L.) can be bred by both self-pollination and cross-pollination techniques. The sunflower head (inflorescence) usually is composed of about 1,000 to 2,000 individual disk flowers joined to a common base (receptacle). The flowers around the circumference are ligulate ray flowers with neither stamens nor pistil. The remaining flowers are hermaphroditic and protandrous disk flowers.

Natural pollination of sunflower occurs when flowering starts with the appearance of a tube partly exerted from the sympetalous corolla. The tube is formed by the five syngenesious anthers, and pollen is released on the inner surface of the tube. The style lengthens rapidly and forces the stigma through the tube. The two lobes of the stigma open outward and are receptive to pollen but out of reach of their own pollen initially. Although this largely prevents self-pollination of individual flowers, flowers are exposed to pollen from other flowers on the same head by insects, wind and gravity.

Promising advanced breeding lines are thoroughly tested and compared to appropriate standards inenvironments representative of the commercial target area(s) for three years at least. The best lines are candidates for new commercial cultivars; those still deficient in a few trans are used as parents to produce new populations for further selection.

These processes, which lead to the final step of marketing and distribution, usually take from eight to 12 generations from the time the first cross is made. Therefore, development of new cultivars is a time-consuming process that requires precise forward planning, efficient use of resources, and a minimum of changes in direction.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

Each year, the plant breeder selects the germplasm to advance to the next generation. This germplasm is grown under unique and different geographical, climatic and soil conditions, and further selections are then made, during and at the end of the growing season. The inbred lines that are developed are unpredictable. This unpredictability arises because the breeder's selection occurs in unique environments, with no control at the DNA level (using conventional breeding procedures), and with millions of different possible genetic combinations being generated. A breeder of ordinary skill in the art cannot predict the final resulting lines that will ultimately be developed, except possibly in a very gross and general fashion. The same breeder cannot produce the same line twice by using the exact same original parents and the same selection techniques. This unpredictability results in the expenditure of large research monies to develop a superior new sunflower inbred line.

Descriptions of breeding methods that are commonly used for different traits and crops can be found in one of several reference books (e.g., Allard, 1960; Simmonds, 1979; Sneep et al., 1979; Fehr, 1987).

A mutant sunflower was reported by Heaton et al., but the locus of that mutation is unknown. T. C. Heaton, et al., 1981, “Rapid Conversion of Maintainer Lines to Cytoplasmic Sterility,” Proceedings Sunflower Forum and Research Workshop, p.2.3.

### BRIEF SUMMARY OF THE INVENTION

The subject invention relates in part to the discovery of a spontaneous sunflower mutation. The subject invention involves an “early” mutation and related inbred/hybrid development. The subject invention further provides a single dominant gene that confers earliness in sunflower inbred isolines and near isogenic hybrids. There is no known prior teaching or suggestion of this gene's utility for hybrid development in the industry. The subject invention also provides a new and distinctive sunflower inbred tine designated H120R.

The invention includes seeds that possess this mutated gene, plants produced by growing these seeds, and progeny thereof that possess this mutated gene and the associated earliness trait. The subject invention also includes methods for producing such sunflower seeds and plants, including inbreds and hybrids. Such plants can be produced by, for example, crossing such an inbred line with itself or with another sunflower line. The invention further relates to such plants and methods for producing such sunflower plants further containing in their genetic material one or more transgenes. Parts of a sunflower plant of the present invention are also provided, such as e.g., pollen obtained from an inbred plant and an ovule of the inbred plant, wherein such parts comprise an early maturity gene of the subject invention.

The subject invention can significantly reduce the phenophase emergence flowering without affecting the filling period. This invention can also significantly increase the IC. This invention can also be used to convert very late, elite inbreds in earlier iso-lines for other geographies that require shorter maturity. This invention can also be used to increase density tolerance and for intercroppng.

### BRIEF DESCRIPTION OF THE FIGURES

FIG. 1 shows a photo of the H120R isoline showing flower development comparisons between the late Argentine line H120R and its early mutated version.

FIG. 2 shows relationships between (A) leaf area index and (B) the proportion of the incident radiation which is intercepted by the crop (Qd) and the time from first anthesis for genotypes X223 (MG2em) and MG2. Vertical bars indicate standard deviation, when larger than the symbol.

FIG. 3 shows bi-lineal relationship between seed weight and time from first anthesis for genotypes X223 (MG2em) and MG2. Vertical bars indicate standard deviations, when larger than the symbol.

FIG. 4 shows bi-lineal relationship between harvest index (corrected for synthesis costs) and time from first anthesis for genotypes X223 (MG2em) and MG2. Vertical bars indicate standard deviations, when larger than the symbol.

FIG. 5 shows a genetic map of a major locus for the early flowering (EF) gene. See Example 8.

FIG. 6 illustrates a strategy for marker development.

FIG. 7 shows markers flanking the early flowering gene of the subject invention.

FIG. 8 illustrates an accelerated introgression strategy.

### BRIEF DESCRIPTION OF THE SEQUENCES

SEQ ID NOs:1-60 are forward and reverse primers as discussed in Example 8.

SEQ ID NO:61 is the HA1805 forward primer.

SEQ ID NO:62 is the HA1805 reverse primer.

SEQ ID NO:63 is a genomic sequence comprising two single nucleotide polymorphism (SNP) loci as discussed in Example 9; SEQ ID NO:82 shows the SNPs as found in the early flowering/early maturing gene/line.

SEQ ID NO:64 is a forward primer for amplifying the “R” SNP locus.

SEQ ID NO:65 is a reverse primer for amplifying the “R” SNP locus.

SEQ ID NO:66 is a probe comprising the early-maturing nucleotide/polymorphism at the R locus.

SEQ ID NO:67 is a probe comprising the wild-type nucleotide at the R locus.

SEQ ID NOs:68-81 are marker sequences discussed in Example 9.

SEQ ID NO:82 is a genomic sequence comprising two single nucleotide polymorphisms (SNPs) as discussed in Example 9; SNPs as found in the early flowering/early maturing gene/line occur at residues 65 (the “Y” locus) and 125 (the “R” locus).

### DETAILED DESCRIPTION OF THE INVENTION

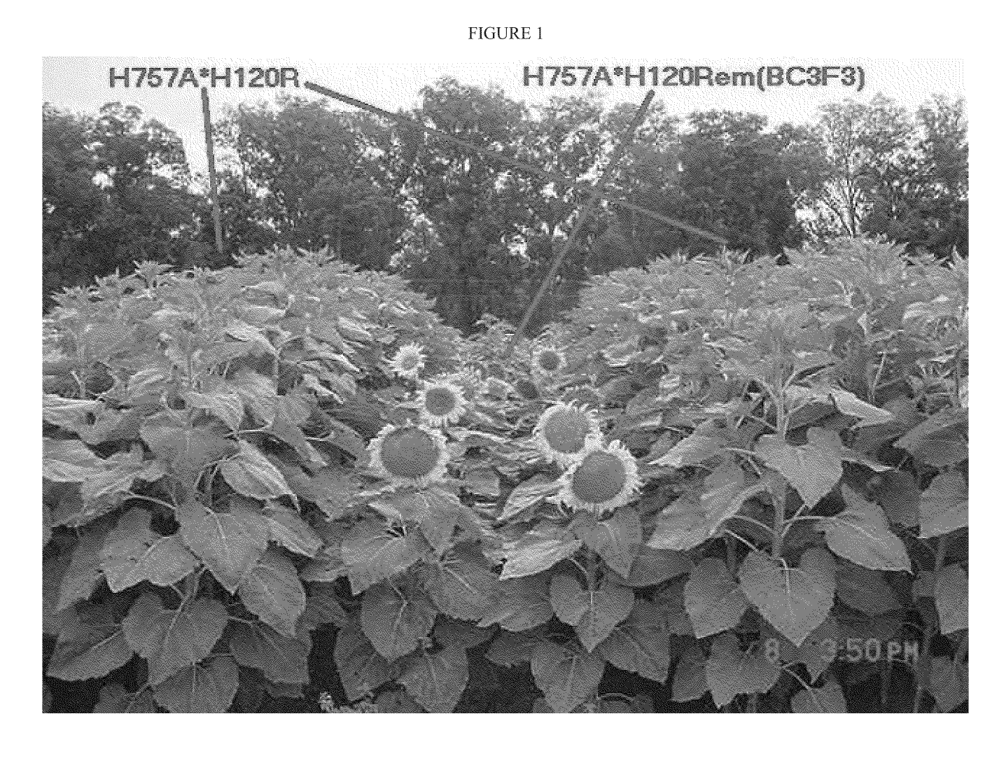

The subject invention relates in part to the discovery of a spontaneous sunflower mutation. The subject invention involves an “early” mutation and related inbred/hybrid development. The subject invention further provides a single dominant gene that confers earliness in sunflower inbreds and hybrids, including inbred isolines and near isogenic hybrids. There is no known prior teaching or suggestion of this gene's utility for hybrid development in the industry. The subject invention also provides a new and distinctive sunflower inbred line designated H120R. The mutation was discovered in nursery row 2290141 of a H792A inbred increase block. FIG. 1 is a photo of the H120R isoline showing flower development comparisons between the late Argentine line H120R and its early mutated version.

This gene was originated by natural mutation in a sunflower breeding population. It was initially used to create hyper-early versions of early inbreds pursuing adaptation to short maturity regions. Later on its potential use to normalize hyper-late inbreds was understood and applied.

Inheritance of the subject traits conferred by the subject gene appears to be qualitative (single and incomplete dominance). The effect is seen as clearly dominant, but there are some indications of “gene dosage” effects.

Insertion of this gene (by backcrossing, for example) will allow the direct use of converted late sunflower inbreds in earlier environments. It can also be used for transgenic research and development in other crops.

The gene could allow late genotypes with desirable traits, quantitative and qualitative, to be moved into earlier (shorter season) environments. The same concept could be applied for the transgenic development of other crops. That is, this trait can also be bred or otherwise introduced into other, non-sunflower crops. For example, with successful applications, tropical corn germplasm could be made available for use in the central U.S. corn belt, for example. In addition, central corn belt germplasm could be moved north.

The early gene may also have utility as an aid in backcrossing traits, some examples of which include cytoplasmic male sterility or imidazitinone (IMI) resistance. If the heterozygote early flowering backcross F1 progeny are selected with the desired donor trait, the conversion cycle could be shortened. (Selling would occur at the final stages of conversion when the desired maturity is selected.)

This gene can be transferred to other sunflower inbreds by the backcross method of breeding. Only one converted inbred is required to develop a hybrid conferring earlier maturity.

The early mutation gene appears to confer relatively proportionate decreases in days to flower, and thus maturity, for a wide range of conventional recurrent parent maturities. Proportionate flowering/maturity modifications are desirable, as it is undesirable for all inbreds, and thus hybrids, to mature in the same number of days for a restricted marketing area.

The invention includes seeds that possess this mutated gene, plants produced by growing these seeds, and progeny thereof that possess this mutated gene and the associated earliness trait. The subject invention also includes methods for producing such sunflower seeds and plants, including inbreds and hybrids. Such plants can be produced by, for example, crossing such an inbred line with itself or with another sunflower line. The invention further relates to such plants and methods for producing such sunflower plants further containing in their genetic material one or more transgenes. Parts of a sunflower plant of the present invention are also provided, such as e.g., pollen obtained from an inbred plant and an ovule of the inbred plant, wherein such parts comprise an early maturity gene of the subject invention.

“Early Maturity” means a mean time to physiological maturity (where physiological maturity is defined as the time sunflower plant seed fill is complete), which ranges from between about 60 days to about 90 days. In some embodiments, this can be from about 60 days to about 70 days.

“Early Flowering” means a mean time to flowering for a sunflower plant which ranges from between about 48 days to about 66 days. In some embodiments, this can be from about 48 days to about 55 days.

By routine screening, it is expected that EM plants may vary in Early Maturity and Early Flowering by approximately 10%.

Head size (head periphery), dry seed weight and/or yield is statistically the same for EM and for wild-type.

CNE840B is the early mutant conversion of H840B. That is they are genetically the same except CNE840B has the mutation, and H840B does not. CNE840B is a backcross 5 derivation of H840B (as the recurrent parent)×an early mutant donor parent.

As part of this disclosure, at least 2500 seeds of early maturing sunflower line CNE840B, comprising the early maturity gene, have been deposited in accordance with the Budapest Treaty on Oct. 17, 2007, and made available to the public without restriction (but subject to patent rights), with the American Type Culture Collection (ATCC) Manassas, Va. 20110-2209. The deposit has been designated as ATCC Deposit No. PTA-8715. The deposit will be maintained without restriction at the ATCC depository, which is a public depository, for a period of 30 years, or five years after the most recent request, or for the effective life of the patent, whichever is longer, and will be replaced if it becomes nonviable during that period.

The deposited seeds arc part of the subject invention. Clearly, plants can be grown from these seeds, and such plants are part of the subject invention. The subject invention also relates to DNA sequences contained in these plants. Related early maturing progeny thereof, including the use of the parent plants and such progeny plants in crosses, are part of the subject invention. Detection methods and kits, of the subject invention, can be directed to identifying any of the deposited and/or progeny lines thereof.

In other aspects, the present invention provides regenerable cells, comprising such genes, for use in tissue cultures, for example. The tissue culture will preferably be capable of regenerating plants having the physiological and morphological characteristics of the foregoing sunflower plant, and of regenerating plants having substantially the same genotype as the foregoing inbred sunflower plant. Preferably, the regenerable cells in such tissue cultures will be embryos, pollen, ovules, leaves, stems, cortex, pith, involucral bracts, ray flowers, disk flowers, pappi, achenes, nectaries, interfloral bracts, receptacle, trichomes stigma, anther, style, filament, calyx, pericarp, seed coat, endosperm, embryo, roots, root tips, seeds and the like. Still further, the present invention provides early maturing sunflower plants regenerated from the tissue cultures of the invention.

Days to flower in the early isolines of H418R and H102R were 62 and 66 days, respectively, compared to 68 and 75 days for the recurrent parents. For comparisons involving normal early line conversions to early mutant, at one location, flowering occurred in as few as 35-37 days after planting in Group 1 F3 early mutant derivations (with the gene in Very Early segregating F3 derivations), versus 48 days for the normal (Group 1 derivations) Very Early (Group 1) inbred. At another location, days to flower for the early mutant isolines and its late maturing recurrent parent H840B (Argentine inbred) were 64 vs 80, respectively. Maturity classification changed from the recurrent parent's group 7 (very late) to the early mutant conversion of group 3 (moderately early).

The subject gene can also be stacked with other traits. This can be accomplished in a variety of ways. Cross-breeding with other lines (having other traits) is known in the art. See e.g. CLEARFIELD™ Sunflower (Helianthus annuus) Line X81359. Also, the subject trait and/or other traits can be genetically engineered to obtain a plant comprising the desired combination of traits. For example, ornamental and confection (for human consumption) lines and varieties can be introgressed with the subject earliness gene. See e.g.:

Yue et al. (2007) “Experimenting with marker-assisted selection in confection sunflower germplasm enhancement.” 29th Sunflower Research Workshop, Jan. 10-11, 2007, Fargo, N.D. (available at website sunflowernsa.com/research/research-workshop/documents/Yue_Experiment_Marker_{—}07.pdf).

Miller et at. (2006) “Registration of three low cadmium (HA 448, HA 449, and RHA 450) confection sunflower genetic stocks.” Crop Science. 46:489-490 (Jan. 1, 2006).

“Interspecific hybridisation and cytogenetic studies in ornamental sunflower breeding,” J. Atlagic et al., Australian Journal of Experimental Agriculture 45(1) 93-97, published 21 Feb. 2005.

“Genes for pollen fertility restoration in sunflowers.” Euphytica, Volume 27, Number 2/June, 1978, pp. 617-627.

Some other examples of some traits and lines are in the following patent references:

| U.S. Pat. No. or | | Application |
|---|---|---|
| U.S. application | | Filing Date |
| Ser. No. | Title or subject matter | (if applicable) |
| 61/015,591 | Low Saturated-Fat Sunflower and | 20 Dec. 2007 |
| | Associated Methods | |
| 61/015,576 | Glyphosate Resistant Sunflower and | 20 Dec. 2007 |
| | Associated Methods | |
| USSN 11/245,991 | Sunflower Seed with High Delta- | 7 Oct. 2005 |
| (Published as | Tocopherol Content | |
| 2006/0112450A1) | | |
| 60/721,181 | High Oleic Imidazolinone Resistant | 28 Sep. 2005 |
| | Sunflower | |
| USPNs 4,627,192 | High Oleic Sunflower | |
| and 4,743,402 | | |
| USPN 5,276,264 | Sunflower Products having Lower | |
| | Levels of Saturated Fatty Acids | |
| USPN 6,977,328 | Sunflower Seed having Low | |
| | Saturated Oil Content (also high | |
| | oleic) | |
| USPN 6,956,156 | Inbred Sunflower Line H1063R (also | |
| | high oleic and imidazolinone | |
| | resistant) | |

All patents, patent applications, provisional applications, and publications referred to or cited herein are incorporated by reference in their entirety to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

### Example 1

### Agronomic Testing and Sample Results

This gene was originated by natural mutation in a sunflower breeding population. This gene was initially used to create hyper-early versions of early inbreds pursuing adaptation to short maturity regions. Later on its potential use to normalize hyper-late inbreds was understood and applied.

A set of experiments was carried out with the purpose to initiate characterization of the em gene in sunflower, using the following genotypes:

| | | |
|---|---|---|
| MG2 | H757A*H120R | Wild type |
| X223 | H757A*EM229135R | Early Mutant type |

EM229135R = H120Rem (BC2F7 homozygous)

Some of the highlights are the following:

TABLE 1
Mean values for time to flowering (DtoFLW) and physiological maturity
(DtoPhM), of two sunflower hybrids contrasting in earliness character.

| | | |
|---|---|---|
| Genotype | P | d.m.s |

| Attribute | X223 | MG2 | G | α = 0.10 |
|---|---|---|---|---|
| DtoFLWB (day) | 56* | 67 | 0.00 | 1 |
| DtoPhM (day) | 89 | 99 | 0.11 | 11 |

G: genotype,
d.m.s: significant difference.
*= note reduction in DTF, but same length of seed filling period

TABLE 2
Mean values for leaf area index near of 13 (X223) and 16 (MG2)
days after first anthesis (LAI13/16) and physiological maturity
(LAIMF), onset of leaf (SLS), rate of leaf senescence (LSR)
and proportion of the incident radiation which is intercepted
by the crop near of 13 (X223) y 16 (MG2) days after first anthesis
(Qd13/16) and physiological maturity (QdMF) of two sunflower
hybrids contrasting in earliness character.

| | | |
|---|---|---|
| Genotype | P | d.m.s |

| Attribute | X223 | MG2 | G | α = 0.10 |
|---|---|---|---|---|
| LAI_{13/16} | 1.80* | 3.40 | 0.01 | 0.53 |
| LAI_{PhM} | 1.63* | 2.69 | 0.17 | 0.17 |
| SLS (day) | 32.5 | 30.9 | 0.53 | 7.1 |
| LSR (LAI day-1) | −0.104 | −0.198 | 0.14 | 0.130 |
| Qd_{13/16} | 0.848 | 0.959 | 0.01 | 0.041 |
| Qd_{MF} | 0.836* | 0.903 | 0.24 | 0.132 |

*= note reduction in LAI, with consequent lower light interception ratio

TABLE 3
Agronomic characters and mean values for head diameter
(HEAD_DIAM), petiole length (PET_LENG) and height (HEIGHT)
of two sunflower hybrids contrasting in earliness character.

| | | |
|---|---|---|
| Genotype | P | d.m.s |

| Attribute | X223 | MG2 | G | α = 0.10 |
|---|---|---|---|---|
| HEAD_THICKNESS † | thin | thin | | |
| HEAD_SHAPE †† | flat | flat | | |
| NECK BENDING | 30 | 30 | | |
| (degree) ‡ | | | | |
| HEAD_DIAM (cm) | 16.2 | 18.9 | 0.05 | 2.2 |
| PET_LENG (cm) | 13.1 | 16.3 | 0.00 | 0.46 |
| HEIGHT (m) | 1.18 | 1.88 | 0.00 | 0.15 |

† Head thickness
†† Head shape
‡ Head orientation

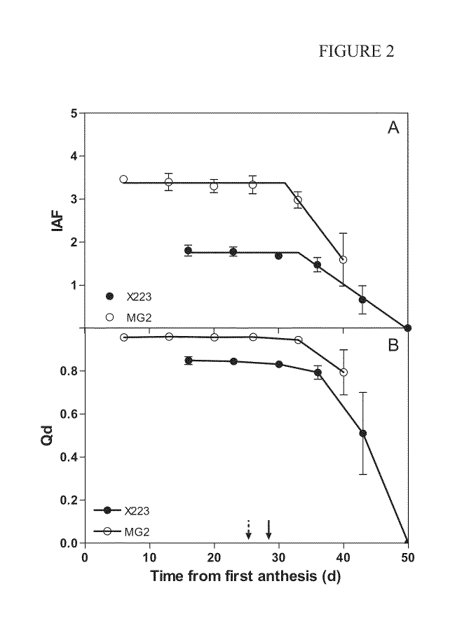

FIG. 2 shows the relationships between (A) leaf area index and (B) the proportion of the incident radiation which is intercepted by the crop (Qd) and the time from first anthesis for genotypes X223 (MG2em) and MG2. Vertical bars indicate standard deviation, when larger than the symbol.

TABLE 4
Mean values for oil yield (OIL YLD), grain number (#GRAINS), grain
weight (GW), rate of grain filling (FR), time from anthesis to the
end of grain filling (FD) and kernel percentage (% E) for the portions
periphery (PER), intermediate (INT) and inner (CEN) of the head and
grain oil concentration (OIL %) of two sunflower hybrids contrasting
in earliness character; *= most significant difference

| | Genotype | | P | d.m.s |
|---|---|---|---|---|
| Attribute | X223 | MG2 | G | α = 0.10 |
| OILYLD (kg/ha) | 1049 | 1374 | 0.15 | 466 |
| #GRAINS m-2 | 6078* | 8109 | 0.10 | 2342 |
| GW_PER (mg) | 70.86 | 65.89 | 0.47 | 18.15 |
| GW_INT (mg) | 60.91 | 57.83 | 0.37 | 8.59 |
| GW_CEN (mg) | 48.40 | 50.79 | 0.33 | 7.73 |
| FR_PER (mg d-1) | 2.21 | 2.66 | 0.15 | 0.63 |
| FR_INT (mg d-1) | 2.16 | 2.61 | 0.53 | 1.97 |
| FR_CEN (mg d-1) | 1.71 | 1.43 | 0.61 | 2.25 |
| FD_PER (day) | 31.1 | 31.2 | 0.96 | 6.8 |
| FD_INT (day) | 33.6 | 32.7 | 0.80 | 10.6 |
| FD_CEN (day) | 35.2 | 40.0 | 0.32 | 15.2 |
| OIL % | 45.8 | 46.6 | 0.60 | 4.01 |

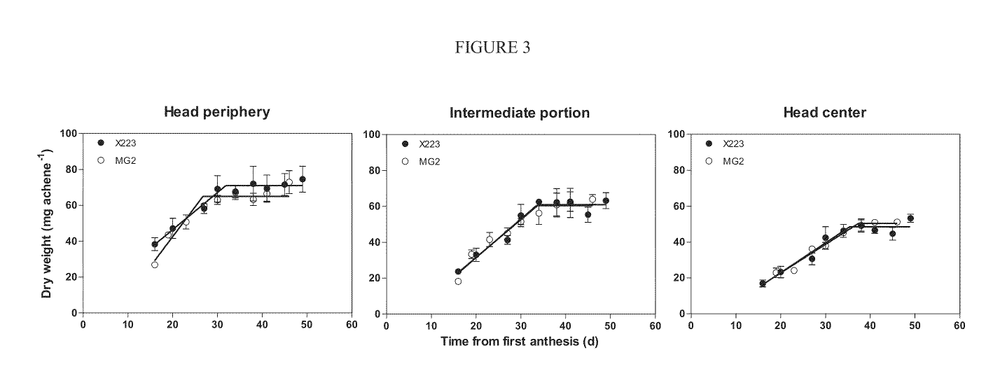

FIG. 3 shows bi-lineal relationship between seed weight and time from first anthesis for genotypes X223 (MG2em) and MG2 planted in Colón 2002/03. Vertical bars indicate standard deviations, when larger than the symbol.

TABLE 5
Mean values for oil-corrected grain yield (YLD), oil-corrected biomass
near of 12 (X223) and 15 (MG2) days after first anthesis (BMco12/15)
and physiological maturity (BMCoMF), production of oil-corrected
biomass (ΔBMco12/15-MF) and daily production of oil-corrected
biomass between 12 (X223) and 15 (MG2) days after first anthesis
and physiological maturity of two sunflower hybrids contrasting
in earliness character. 12/15: days after first anthesis (12 days
X223 and 15 days MG2), MF: physiological maturity.

| | Genotype | | P | d.m.s |
|---|---|---|---|---|
| Attribute | X223 | MG2 | G | α = 0.10 |
| YLD corr by OIL (g m-2) | 378 | 490 | 0.14 | 159 |
| BMco12/15 (g m-2) | 644 | 1277 | 0.00 | 200 |
| BMcoMF (g m-2) | 1014 | 1810 | **0.00 | 251 |
| ΔBMco12/15-MF (g m-2) | 371 | 534 | 0.00 | 52 |
| RBMco12/15-MF (g m-2 day-1) | 21.3 | 33.7 | 0.02 | 5.05 |

**= note that YLD, Biomass, rate, and HI are different

TABLE 6
Mean values for harvest index (HI), rate of daily HI increase
(HIR) and duration of the linear phase of HI increase (HID),
of two sunflower hybrids contrasting in earliness character.

| | | |
|---|---|---|
| Genotype | P | d.m.s |

| Attribute | X223 | MG2 | G | α = 0.10 |
|---|---|---|---|---|
| HI * | 0.526 | 0.466 | 0.01 ** | 0.020 |
| HIR (IC day-1) | 0.0163 | 0.0140 | 0.75 | 0.015 |
| HID (day) | 31.7 | 34.1 | 0.41 | 7.5 |

* Determined as the ratio of oil-corrected grain dry matter to oil-corrected above ground dry matter.

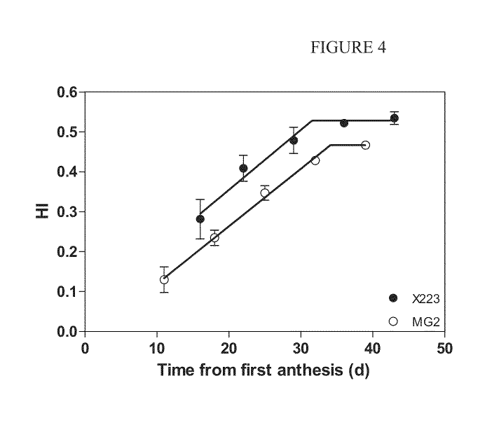

FIG. 4 shows bi-lineal relationship between harvest index (corrected for synthesis costs) and time from first anthesis for genotypes X223 (MG2em) and MG2 planted in Colón 2002/03. Vertical bars indicate standard deviations, when larger than the symbol.

### Example 2

### Characterization of Gene Dominance, Gene Dosage, and Application of the em Gene for Reducing Phenophase Without Affecting the Filling Period

The subject mutation/mutated gene can be used to significantly reduce the phenophase “emergence-flowering” (V1-R5.1), without affecting the subsequent filling period (R5.5-R9), in the sunflower growing cycle; it could be used to convert very late “elite inbreds showing reduced Genotype Environment interaction” in earlier “iso-lines” for other geographies that require shorter maturity.

Two inbreds have already been converted (BC4+) and fixed (H840B and H418R), and one partially converted (BC2F7) but fixed (H120R), The em versions of both H840B and H120R fits perfectly the maturity normally used in North America. These em versions of inbred lines have even been useful to create hybrids of mg 3, being the recurrent inbreds mg 7 and mg 6, respectively. This hybrid performed earlier than 8377NS and near SF270.

H840B was used to make experimental hybrids with very good rust tolerance in the past. They were outstanding in performance but very late and tall. The new em version can be used to re-create those hybrids, and to include it in the “elite collection”, once the cited problems have been removed by the effect of the em gene.

Based on various observations, the gene inheritance appears to be qualitative (single and incomplete dominant). The effect is seen as clearly dominant, hut there are some indications of “gene dosage” effects. If this is true, it would allow creation of iso-hybrids for different maturity groups by using the gene in both hetero or homzygous form, which would expand even more the use of elite germplasm.

A series of experiments (RCBD) have been designed to prove/reject that hypothesis, with the purpose to clearly identify the inheritance mode and gene action, by the study of the following genotypes:

TABLE 7

| Entry Code | Gen | Genotype |
|---|---|---|
| H840B | | Ee |
| H840Bem | | EE |
| H840B/H840Bem | F1 | ee/EE |
| (H840B/H840Bem)@ | F2 | EE; Ee; ee |
| H840B//H840B/H840Bem | BC1F1(−) | ee//ee/EE |

TABLE 8

| Entry Code | Gen | Genotype |
|---|---|---|
| H418R | | Ee |
| H418Rem | | EE |
| H418R/H418Rem | F1 | Ee |
| (H418R/H418Rem)@ | F2 | EE, Ee, ee |
| H418R//H418R/H418Rem | BC1F1(−) | ee//ee/EE |
| H418Rem//H418R/H418Rem | BC1F1(+) | EE//ee/EE |

TABLE 9

| Entry Code | Gen | Genotype |
|---|---|---|
| H840B/H418R | F1 | ee/ee |
| H840B/H418Rem | F1 | ee/EE |
| H840Bem/H418R | F1 | EE/ee |
| H418R/H840Bem | F1 | ee/EE |
| H840Bem/H418Rem | F1 | EE/EE |

In addition to earliness related measurements, pleiothropic effects on traits such as PHGT, HDIAM, SDIAM, #LEAF, etc, will also be measured.

### Example 3

### Use of this Gene to Allow Expansion of the Sunflower Frontiers to Areas with Shorter Growing Seasons and More Limited Water Availability

Due to the significant reduction in that phenophase, the use of this gene can allow expansion of the sunflower frontiers to areas with shorter growing seasons and more limited water availability, by the combined use of elite iso-hybrids from other areas, Because of the pleiothropic effects of the gene on other traits, changes in the crop spatial distribution might be needed to maximize crop productivity. Antecedents such as Sunwheat and Sunola have been extensively tested, but with genetic background limitations.

An experiment has also been setup to study this kind of gene effects, and to quantify the effect of diverse spatial distribution in iso-hybrids, over the growth and developmental parameters and yield components.

TABLE 9

| Entry Code | Gen | Genotype |
|---|---|---|
| H757A/H120R | F1 | (ee/ee) |
| H757A/H120Rem | F1 | (ee/EE) |
| H840A/H418R | F1 | (ee/ee) |
| H840A/H418Rem | F1 | (ee/EE) |

Factorial design with:
Row spacing: 0.70 cms & 0.35 cms
Plant Populations: 35,000, 65,000, and 95,000 pl/ha

### Example 4

### Use of the Subject Gene to Accelerate Introgression of Other Traits

Due to the inheritance of this gene, it is a very powerful toot to accelerate introgression of other traits, by keeping the em gene in heterozygous form along the backcross process. Gene must be introgress in donors or recurrents.

EE XX * ee xx

Ee Xx * ee xx
ee xx Ee Xx * ee xx

ee xx Ee Xx @ → ee XX recovered

### Example 5

### Further Characterization and Sequencing of the Gene

Due to the status of our conversions, the gene could easily be mapped, sequenced, and eventually transformed in a different crop (earliness is very easy to identify).

| | |
|---|---|
| H840Bem*H840B | F1 |
| (H840Bem*H840B)@ | F2 |

### Example 6

### Use of this Gene in Sunflower Hybrid Products

With the development of early mutant inbred isolines nearly complete, the next stage of testing was to determine practical use of this gene in sunflower hybrid products. Limited hybrid testing was done, comparing performance of the early mutant version of the hybrid MG2 against its normal group 6 maturity version and other hybrids of similar Group 3 maturity.

### Example 7

### Insertion of the Gene into Plants

One aspect of the subject invention is the transformation of plants with the subject polynucleotide sequences.

A heterologous promoter region capable of expressing the gene in a plant can be used. Thus, for in planta expression, the DNA of the subject invention is under the control of an appropriate promoter region. Techniques for obtaining in planta expression by using such constructs is known in the art.

A gene of the subject invention can be inserted into plant cells using a variety of techniques that are well known in the art. For example, a large number of cloning vectors comprising a replication system in E. coli and a marker that permits selection of the transformed cells are available for preparation for the insertion of foreign genes into higher plants. The vectors comprise, for example, pBR322, pUC series, M13mp series, pACYC184, etc. Accordingly, the heterologous sequence can be inserted into the vector at a suitable restriction site. The resulting plasmid is used for transformation into E. coli. The E. coli cells are cultivated in a suitable nutrient medium, then harvested and lysed. The plasmid is recovered. Sequence analysis, restriction analysis, electrophoresis, and other biochemical-molecular biological methods are generally carried out as methods of analysis. After each manipulation, the DNA sequence used can be cleaved and joined to the next DNA sequence. Each plasmid sequence can be cloned in the same or other plasmids.

Depending on the method of inserting desired genes into the plant, other DNA sequences may be necessary. If, for example, the Ti or Ri plasmid is used for the transformation of the plant cell, then at least the right border, but often the right and the left border of the Ti or Ri plasmid T-DNA, has to be joined as the flanking region of the genes to be inserted. The use of T-DNA for the transformation of plant cells has been intensively researched and sufficiently described in EP 120 516; Hoekema (1985) In: The Binary Plant Vector System, Offset-durkkerij Kanters B. V., Alblasserdam, Chapter 5; Fraley et al., Crit. Rev. Plant Sci. 4:1-46; and An et al. (1985) EMBO J. 4:277-287.

Once the inserted DNA has been integrated in the genome, it is relatively stable there and, as a rule, does not come out again. It normally contains a selection marker that confers on the transformed plant cells resistance to a biocide or an antibiotic, such as kanamycin, G 418, bleomycin, hygromycin, or chloramphenicol, inter alia. The individually employed marker should accordingly permit the selection of transformed cells rather than cells that do not contain the inserted DNA.

A large number of techniques are available for inserting DNA into a plant host cell. Those techniques include transformation with T-DNA using Agrobacterium tumefaciens or Agrobacterium rhizogenes as transformation agent, fusion, injection, biolistics (microparticle bombardment), or electroporation as well as other possible methods. If Agrobacteria are used for the transformation, the DNA to be inserted has to be cloned into special plasmids, namely either into an intermediate vector or into a binary vector. The intermediate vectors can be integrated into the Ti or Ri plasmid by homologous recombination owing to sequences that are homologous to sequences in the T-DNA. The Ti or Ri plasmid also comprises the vir region necessary for the transfer of the T-DNA. Intermediate vectors cannot replicate themselves in Agrobacteria. The intermediate vector can be transferred into Agrobacterium tumefaciens by means of a helper plasmid (conjugation). Binary vectors can replicate themselves both in E. coli and in Agrobacteria. They comprise a selection marker gene and a linker or polylinker which are framed by the right and left T-DNA border regions. They can be transformed directly into Agrobacteria (Holsters et al. [1978]Mol. Gen. Genet. 163:181-187). The Agrobacterium used as host cell is to comprise a plasmid carrying a vir region. The vir region is necessary for the transfer of the T-DNA into the plant cell. Additional T-DNA may be contained. The bacterium so transformed is used for the transformation of plant cells. Plant explants can advantageously be cultivated with Agrobacterium tumefaciens or Agrobacterium rhizogenes for the transfer of the DNA into the plant cell. Whole plants can then be regenerated from the infected plant material (for example, pieces of leaf, segments of stalk, roots, but also protoplasts or suspension-cultivated cells) in a suitable medium, which may contain antibiotics or biocides for selection. The plants so obtained can then be tested for the presence of the inserted DNA. No special demands are made of the plasmids in the case of injection and electroporation. It is possible to use ordinary plasmids, such as, for example, pUC derivatives.

The transformed cells grow inside the plants in the usual manner. They can form germ cells and transmit the transformed trait(s) to progeny plants. Such plants can be grown in the normal manner and crossed with plants that have the same transformed hereditary factors or other hereditary factors. The resulting hybrid individuals have the corresponding phenotypic properties.

### Example 8

### Additional Information Regarding the Early Flowering Mutation Gene

Characterization. Table 10 shows significant earlier flowering and shorter heights in the homozygous converted parents versus their normal recurrent parents. Table 11 conversions in various heterozygous backcross F1 stages of development indicate a mostly dominant gene action (incomplete dominance). The first 3 pairs of hybrid comparisons in Table 12 show additional supportive evidence of dominance conferred by the early mutation gene. The last two comparisons between homozygous and heterozygous iso-hybrids indicate a possible dosage effect of the gene—the gene in both parents may be earlier than the gene in one parent, difference depending on pedigree. Given the dominant nature of the gene, introgression into elite parents is easily accomplished by traditional backcrossing by selecting for early segregates in the BCnF1 generations for further backcrossing to the elite recurrent parents. Once fully introgresssed, the BCnF1 is selfed to select for individual homozygote EM segregates in the BCnF2 population. Presence of homozygosity can be observed for subsequent BCnF3 family rows.

TABLE 10
Parent days to flower and height comparisons between
homozygous EM conversions and recurrent parent.

| Name | DTF | Ht (in) |
|---|---|---|
| EM BC5 H840A | 65 | 63 |
| H840A | 85 | 68 |
| EM BC4 CN2922R | 53 | 46 |
| CN2922R | 71 | 64 |
| EM BC4 H120R | 53 | 46 |
| H120R | 67 | 61 |
| EM BC4 H535A | 54 | 57 |
| H535A | 66 | 63 |
| EM H1063R | 44 | 35 |
| H1063R | 58 | 53 |
| EM BC5 H418R | 49 | 35 |
| H418R | 59 | 46 |

TABLE 11
Days to flower comparisons between heterozygous
BCF1 conversions and recurrent parent line.

| Name | DTF |
|---|---|
| EM BC2F1 ON2343B | 54 |
| ON2343B | 61 |
| EM BC1F1 HO00537B | 58 |
| HO00537B | 71 |
| EM BC1F1 CN2798B | 63 |
| CN2798B | 68 |
| EM BC1F1 HO00587B | 59 |
| HO00587B | 65 |
| EM BC1F1 CN1907R | 56 |
| CN1907R | 73 |
| EM BC1F1 ON1938R | 59 |
| ON1938R | 71 |
| EM BC1F1 ONN947R | 56 |
| ONN947R | 62 |
| EM BC3F1 HOU98510R | 51 |
| HOU98510R | 59 |
| EM BC3F1 MOC0666R | 57 |
| MOCO666R | 62 |
| EM BC2F1 H324B | 38 |
| H324B | 49 |

TABLE 12
Days to flower and height comparisons between hybrids with
one, two, or no EM parents. Two (consecutive) years.

| Name | DTF | Ht (in) |
|---|---|---|
| H757A × EM H120R | 60 | na |
| H757A × H120R | 78 | na |
| H757A × EM H418R | 57 | na |
| H757A × H418R | 65 | na |
| CN1703A × EM H1063R | 56 | 42 |
| CN1703A × H1063R | 65 | 68 |
| EM 840A × EM CN2922R | 61 | 73 |
| EM 840A × CN2922R | 66 | 84 |
| EM 840A × EM H1063R | 56 | 58 |
| H840A × EM H1063R | 57 | 60 |

Tables 13 and 14 show the additional pleiotropic effects of the early flowering mutant. Raw data in Table 13 show a general reduction in leaf number, width, and length; shorter petioles; smaller head size; and shorter plant heights. This appears to be the reason for Table 14 results which show significantly less leaf area index (for 13 and 16 days post flowering), light interception ratio (13 and 16 days post flowering), and biomass (13 and 16 days post flower, and at physiological maturity) for the EM hybrid compared to the normal hybrid. However, the harvest index ratio (grain matter/total above ground plant dry matter) is significantly more for the EM hybrid. This is good for higher population uses, which is discussed in point 3 below under gene utility.

TABLE 13
Plant trait measurement comparisons between
EM and normal types.

| | | leaf | leaf | petiole | head | plant |
|---|---|---|---|---|---|---|
| | leaf | width | length | length | width | ht |
| plant type | no. | (in.) | (in.) | (in.) | (in.) | (in.) |
| EM Inbreds (4) | 25.5 | 8.5 | 9 | 5.2 | 5.2 | 50.5 |
| Normal Inbreds | 33 | 9.3 | 9.7 | 5.6 | 7 | 63.8 |
| EM Hybrids (2) | 28.5 | 12.1 | 12.1 | 5.6 | 10 | 55.9 |
| Normal Hybrids | 34.5 | 12 | 13.2 | 6.1 | 12 | 70.4 |

TABLE 14
Leaf area index (LAI), light interception ratio (Qd), and biomass
(BM) for EM and normal hybrid 13 and 16 days after first anthesis and
physiological maturity (MF); and harvest index (HI) comparison.

| | | LAI | LAI | Qd | Qd | BM | BM | |
|---|---|---|---|---|---|---|---|---|
| Hybrid | | 13/16 | MF | 13/16 | MF | 13/16 | MF | HI* |
| H757A × EM H120R | | 1.8 | 1.63 | 0.85 | 0.84 | 644 | 1014 | 0.526 |
| H757A × H120R | | 3.4 | 2.69 | 0.96 | 0.90 | 1277 | 1810 | 0.466 |
| | P | 0.01 | 0.17 | 0.01 | 0.24 | 0.00 | 0.00 | 0.01 |

*Determined as me ratio of grain dry matter to above ground dry matter.

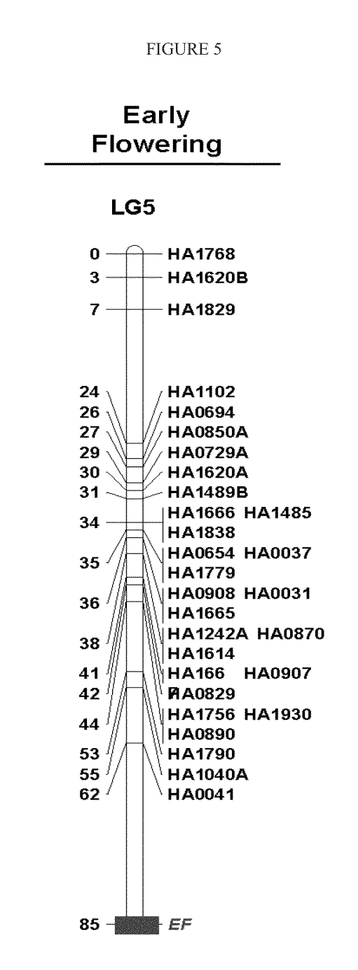

A major locus for the early flowering gene was mapped on one end of linkage group 5 using microsatellite or SSR (Simple Sequence Repeat) markers and flowering data of F3 families from the cross MOC0666R×CNE418.312. See FIG. 5.

The maps in sunflower are usually referred to by linkage groups. Linkage group 5 corresponds to the maps published by Dr. Steve N. Knapp's group. See See Yu et al., (2003) “Towards a saturated molecular genetic linkage map for cultivated sunflower,” Crop Sci. 43:367-387; and Tang et al. (2002) “Simple sequence repeat map of the sunflower genome,” Theor Appl Genet 105: 1124-1136. Linkage group numbers of maps developed by European scientists are different from the ones developed by Dr. Knapp's group. The chromosome numbers have not been defined in sunflower yet.

Following are primer sequences and map positions of the SSR markers mapped on linkage group 5, where the early flowering locus (EF) is mapped.

| | Map | | |
|---|---|---|---|
| | Position | | |
| Marker | (cM) | Forward Primer Sequence | Reverse Primer Sequence |
| HA1768 | 0 | AAATCCACAAGGATGCTCAATC | GGAGATCATACAAAGCGTTATCGT |
| | | SEQ ID NO: 1 | SEQ ID NO: 2 |
| HA1620B | 3 | HEX-TTTCGTGATGGTGATTGATGATT | CAGCAACTCTGACCGTTTCATTA |
| | | SEQ ID NO: 3 | SEQ ID NO: 4 |
| HA1829 | 7 | CATTGAGGACGAGAAGCCAGT | GTTCCGTACCCTGTTTGAGCTT |
| | | SEQ ID NO: 5 | SEQ ID NO: 6 |
| HA1102 | 24 | 7 TGTTCACAGCTCCCGTCTAA | CACACACACAACAACCTGACC |
| | | SEQ ID NO: 7 | SEQ ID NO: 8 |
| HA0694 | 26 | GCCGTGAATAATGGGATTGA | GATTGGGTCAGCTTGTGTGA |
| | | SEQ ID NO: 9 | SEQ ID NO: 10 |
| HA0850A | 27 | CCCTGGAGTGTATGTCCGTTA | ATCCGTCTGCTGCCTAATCC |
| | | SEQ ID NO: 11 | SEQ ID NO: 12 |
| HA0729A | 29 | TGAAACGTAGTAACCTGCCAAA | TTGGACGACCTCGGTATCTT |
| | | SEQ ID NO: 13 | SEQ ID NO: 14 |
| HA1620A | 30 | HEX-TTTCGTGATGGTGATTGATGATT | CAGCAACTCTGACCGTTTCATTA |
| | | SEQ ID NO: 15 | SEQ ID NO: 16 |
| HA1489B | 31 | CTTATTCCAAGGACGCATAGTCG | CGATGGTATGATTCTCGACGTTA |
| | | SEQ ID NO: 17 | SEQ ID NO: 18 |
| HA1666 | 34 | FAM-ATTTCACCCTCACTCCCACAC | TACCGGCTGGATATGGAGAAT |
| | | SEQ ID NO: 19 | SEQ ID NO: 20 |
| HA1485 | 34 | GGGAAGTGGGCTTGTCTATGTAT | AACACACCGAAATCACCTATGAA |
| | | SEQ ID NO: 21 | SEQ ID NO: 22 |
| HA1838 | 34 | AGAGGAATGAGATCGGGTTGAT | GTGGGACAACTCAGCAACGTC |
| | | SEQ ID NO: 23 | SEQ ID NO: 24 |
| HA0037 | 35 | GAACATGGCCATAACTCATAGACG | CCTTCGACCCAACATC |
| | | SEQ ID NO: 25 | SEQ ID NO: 26 |
| HA0654 | 35 | ACGCACATGAGAGAGAAAGAG | ACCTTCGACCCAACATCAAG |
| | | SEQ ID NO: 27 | SEQ ID NO: 28 |
| HA1779 | 35 | ATTTGTTCCTGGTTCGGTATCC | CATGTCTGATCTTCGGGACTTC |
| | | SEQ ID NO: 29 | SEQ ID NO: 30 |
| HA0031 | 36 | CTCACGAAACTCTTCATGCTG | CTCTCACACTTACTGAAC |
| | | SEQ ID NO: 31 | SEQ ID NO: 32 |
| HA1665 | 36 | FAM- | AACTTCCAATGTTCTCCAACCAT |
| | | CCTAAGGGGATGAATTCTCTTTC | SEQ ID NO: 34 |
| | | SEQ ID NO: 33 | |
| HA0908 | 36 | TTGTCTTCATCTGCGTGTGA | TTGCTGTTGTTGATCGGTGT |
| | | SEQ ID NO: 35 | SEQ ID NO: 36 |
| HA0870 | 38 | GTGCGTTGGCTCTTATGGAT | AGTGATGGCATTCCCAATTT |
| | | SEQ ID NO: 37 | SEQ ID NO: 38 |
| HA1614 | 38 | HEX-GTGATCCGAGTTGTGGATGTTC | GTTAGATGGCAACCCAAGTGAT |
| | | SEQ ID NO: 39 | SEQ ID NO: 40 |
| HA1242A | 38 | GGTGATGATGGAGGAGCAACTG | CACTCAACCATTGTTCTCCCAC |
| | | SEQ ID NO: 41 | SEQ ID NO: 42 |
| HA1667 | 41 | FAM-GAACTCCGGTTAGTCTTCCGAC | GCAATTAAGTCTGCGTTTCAGTTT |
| | | SEQ ID NO: 43 | SEQ ID NO: 44 |
| HA0907 | 41 | CATGAACATCGCCAATTCAG | TGCAAGGAACCATCAGAATC |
| | | SEQ ID NO: 45 | SEQ ID NO: 46 |
| HA0829 | 42 | TTGTCATGTATGGGCTTTGG | ATCCAACAGGTGTGCGGAAT |
| | | SEQ ID NO: 47 | SEQ ID NO: 48 |
| HA0890 | 44 | CACTTCATCCTCTCCCTCCTT | GGCGTGTGTGTTGGGTTATT |
| | | SEQ ID NO: 49 | SEQ ID NO: 50 |
| HA1756 | 44 | ACACGAGTCCCAACCTGAATG | ACCTGAAATGCAAATCTCTACAGG |
| | | SEQ ID NO: 51 | SEQ ID NO: 52 |
| HA1930 | 44 | TAGGCAATAACTTTGGGCGAAT | CCTGAAATGCAAATCTCTACAGG |
| | | SEQ ID NO: 53 | SEQ ID NO: 54 |
| HA1790 | 53 | TCCCCAAACTTGCGTGTAGGT | CATTACAAACCACAGCTCCTTCC |
| | | SEQ ID NO: 55 | SEQ ID NO: 56 |
| HA1040A | 55 | CCTGGAACTGAACCGAGAAC | GCCGTGAAACAGAGAGAGGA |
| | | SEQ ID NO: 57 | SEQ ID NO: 58 |
| HA0041 | 62 | CTAGCAACCAACCTCATTG | GTCTCCTTCTCTTTCTCGGC |
| | | SEQ ID NO: 59 | SEQ ID NO: 60 |
| EF | 85 | | |

Each primer pair corresponds to one marker on the map. These primers were used to amplify the DNA from two parents (one is early flowering, the other is normal flowering) of the mapping population. Each of them amplified the DNA fragments polymorphic between the two parents. Then these primers were used to amplify the individual plants of the mapping population, from which the map was constructed.

### Gene Utility

1) The gene could be used to convert later maturing elite inbreds to earlier iso-lines for other geographies or cultural practices requiring earlier maturing hybrids. Thus, one beneficial consequence is an expanded genetic base and versatility created for breeders. Table 6 results show utility of this concept. The female and male inbreds H840A and CN2922R are very late maturing lines adapted to central to north Argentina for development of group 6-8 hybrids. H535A is a group 6 female used to make late hybrids. H1063R is medium maturing male for group 2-5 hybrid development. Testcrosses of their EM conversions are provided in Table 6. Especially noteworthy, results are shown by the EM 2922R testcrosses—5 of 6 EM 2922R hybrids made group 2 hybrids. Results show very competitive results with the ON3403A testcrosses against the normal group 2, 3, 4, and 5 checks.

TABLE 15
Early mutant and conventional hybrid comparisons from 2006. 3 locations, 6 reps.

| | Fem | Mal | Hyb | Hyb | Ap | Ht | Hlth | H2O | Yld | Oil | OPA | 18:1 |
|---|---|---|---|---|---|---|---|---|---|---|---|---|
| Name | Flr | Flr | Flr | Mat | (1-9) | (in.) | (1-9) | (%) | (lb/ac) | (%) | (lb/ac) | (%) |
| 8N510(CN1703A/ | 65 | 59 | 65 | 5 | 6 | 68 | 6 | 12.2 | 2625 | 46.4 | 1218 | 67.6 |
| H1063R) | | | | | | | | | | | | |
| ON3403A/EM | 64 | 55 | 58 | 2 | 6 | 65 | 6 | 10.5 | 2618 | 46.0 | 1205 | 53.1 |
| BC4 CN2922R | | | | | | | | | | | | |
| 8H350DM (H251A/ | 61 | 60 | 60 | 3 | 5 | 70 | 5 | 11.5 | 2538 | 46.7 | 1187 | 90.8 |
| OND163R) | | | | | | | | | | | | |
| 8N251 (H251A/ | 61 | 55 | 59 | 2 | 6 | 67 | 6 | 10.8 | 2478 | 51.2 | 1268 | 73.1 |
| CN1229R) | | | | | | | | | | | | |
| ON3403A/EM | 64 | 56 | 60 | 2 | 6 | 65 | 6 | 10.6 | 2371 | 46.1 | 1092 | 56.2 |
| BC1 CN2922R | | | | | | | | | | | | |
| EM BC5 H840A/ | 65 | 71 | 66 | 6 | 6 | 85 | 8 | 13.1 | 2327 | 39.4 | 918 | 19.4 |
| CN2922R | | | | | | | | | | | | |
| EM BC5 H840A/ | 65 | 57 | 63 | 5 | 6 | 76 | 8 | 12.8 | 2304 | 43.1 | 992 | 62.3 |
| HO207746R | | | | | | | | | | | | |
| EM BC3 H535A/ | 57 | 63 | 58 | 3 | 6 | 62 | 6 | 11.1 | 2302 | 44.3 | 1020 | 72.4 |
| HOU98510R | | | | | | | | | | | | |
| 8N453DM | 65 | 60 | 61 | 4 | 6 | 65 | 6 | 11.9 | 2292 | 52.4 | 1202 | 73.4 |
| (CN2343A/ | | | | | | | | | | | | |
| OND163R) | | | | | | | | | | | | |
| EM BC5 H840A/ | 65 | 55 | 61 | 4 | 7 | 73 | 7 | 11.5 | 2250 | 45.0 | 1012 | 19.8 |
| EM BC4 CN2922R | | | | | | | | | | | | |
| H251A/EM BC4 | 61 | 56 | 57 | 2 | 6 | 56 | 5 | 10.4 | 2220 | 45.7 | 1014 | 67.0 |
| CN2922R | | | | | | | | | | | | |
| EM BC3 H535A/ | 57 | 71 | 57 | 3 | 5 | 61 | 6 | 10.9 | 2191 | 45.7 | 1000 | 27.7 |
| CN1229R | | | | | | | | | | | | |
| EM BC3 H535A/ | 57 | 60 | 57 | 2 | 4 | 58 | 5 | 10.2 | 2119 | 48.7 | 1032 | 75.3 |
| OND163R | | | | | | | | | | | | |
| EM BC2 H535A/ | 57 | 56 | 58 | 2 | 6 | 59 | 5 | 10.5 | 2108 | 43.4 | 916 | 26.1 |
| EM BC2 CN2922R | | | | | | | | | | | | |
| H251A/EM BC1 | 61 | 56 | 57 | 2 | 5 | 53 | 5 | 10.9 | 2092 | 47.4 | 992 | 46.9 |
| CN2922R | | | | | | | | | | | | |
| EM BC3 H535A/ | 57 | 65 | 59 | 3 | 6 | 64 | 8 | 11.6 | 1985 | 44.7 | 887 | 64.9 |
| MOC0666R | | | | | | | | | | | | |
| SF270 (C8283A/ | 58 | 59 | 58 | 2 | 5 | 54 | 5 | 10.8 | 1974 | 44.2 | 873 | 38.5 |
| 687R) | | | | | | | | | | | | |
| EM BC2 H535A/ | 57 | 59 | 58 | 2 | 4 | 63 | 6 | 10.8 | 1970 | 45.0 | 887 | 64.5 |
| H1063R | | | | | | | | | | | | |
| H535A/EM | 68 | 46 | 56 | 2 | 4 | 53 | 5 | 10.6 | 1780 | 50.1 | 892 | 63.9 |
| H1063R | | | | | | | | | | | | |
| CN1703A/EM | 65 | 46 | 56 | 2 | 5 | 42 | 5 | 10.4 | 1574 | 43.3 | 682 | 63.9 |
| H1063R | | | | | | | | | | | | |
| EM BC5 H840A/ | 65 | 46 | 56 | 2 | 4 | 58 | 6 | 10.8 | 1416 | 46.2 | 654 | 64.4 |
| EM H1063R | | | | | | | | | | | | |
| H840A/EM | 85 | 46 | 57 | 3 | 4 | 60 | 5 | 11.6 | 1334 | 46.1 | 616 | 65.1 |
| H1063R | | | | | | | | | | | | |
| avg | | | 59 | | 5.6 | 62 | 5.6 | 11.2 | 2130 | 46.2 | 961 | 56.8 |
| CV | | | | | | | | | 20.6 | | | |

2) The gene could be used to make ultra early flowering/maturing plants for genetic studies due to short lifecycle. The BC2F1 conversion of H324B (see bottom of Table 11) shows this potential (flowering in 38 days relative to 49 days of its group 1 recurrent parent H324B).

3) The genes pleiotropic effects—reduced biomass (reduced leaf canopy, height) but higher harvest index—makes hybrids favorable to high density populations to improve yields and compete against normal later maturing hybrids. Table 16 shows this concept. All EM H1063R hybrids planted at 36,000 plants per acre yielded higher than the same hybrids planted at 18,000!. The EM hybrids are significantly earlier flowering and have less seed harvest moisture than 8N251 and 8N270 group 2 check hybrids. These very early hybrids could be marketed for late planting dates or double cropping after wheat. Additional studies will be conducted using narrower rows with the higher plant densities.

TABLE 16
Performance results of EM hybrids and checks planted at 18,000
and 36,000 plants per acre in 30 inch rows.

| | fem | | | | | OIL | | |
|---|---|---|---|---|---|---|---|---|
| Name | flr | FLR | MST | TW | YLD | % | OPA | OLE |
| 8N251-HPOP | | 56 | 24.8 | 27.6 | 1779 | 52.3 | 930 | 65.2 |
| 8N251 | | 56 | 27.2 | 27.9 | 1778 | 52.3 | 930 | 65.2 |
| ON3403A × EM H1063R-HPOP | 64 | 48 | 19.8 | 28.5 | 1885 | 47.7 | 898 | 90.2 |
| 8N270 | | 55 | 27.9 | 28.2 | 1978 | 44.2 | 874 | 81.6 |
| 8N270-HPOP | | 55 | 23.8 | 27 | 1955 | 44.2 | 864 | 81.6 |
| CN3351A × EM H1063R-HPOP | 62 | 47 | 15.4 | 27.5 | 1795 | 48.0 | 862 | 64.5 |
| CN2343A × EM H1063R-HPOP | 62 | 47 | 16.1 | 28.3 | 1829 | 44.5 | 813 | 63.9 |
| H251A × EM H1063R-HPOP | 61 | 45 | 13.9 | 25.5 | 1863 | 43.4 | 809 | 86.8 |
| CN2343A × EM H1063R | 62 | 47 | 21.5 | 30.5 | 1639 | 44.5 | 729 | 63.9 |
| ON3403A × EM H1063R | 64 | 48 | 20.4 | 26.4 | 1526 | 47.7 | 727 | 90.2 |
| CN3351A × EM H1063R | 62 | 47 | 19.4 | 27.9 | 1499 | 48.0 | 720 | 64.5 |
| H251A × H1063R | 61 | 45 | 17.8 | 26.6 | 1512 | 43.4 | 657 | 86.6 |
| AVERAGE | | 49.7 | 20.7 | 27.7 | 1753 | 46.7 | 818 | 75.4 |
| CV | | | | | 14.6 | | | |

4) The gene can become a powerful tool to accelerate introgression of other traits by keeping the early mutant gene in heterozygous form during the backcross process. In the example below, the EM gene has been introgressed into the donor parent with desired gene—indicated by the underlined genotype. The recurrent parent is indicated in bold.

| | |
|---|---|
| Start: | EE XX * ee xx |

| | |
|---|---|
| BC0 | Ee Xx * ee xx |
| BC1 | ee xx Ee xx Ee Xx * ee xx |

| | |
|---|---|
| BC2 − BCn | ee xx Ee xx Ee Xx * ee xx |

BCnF2 self to recover ee XX

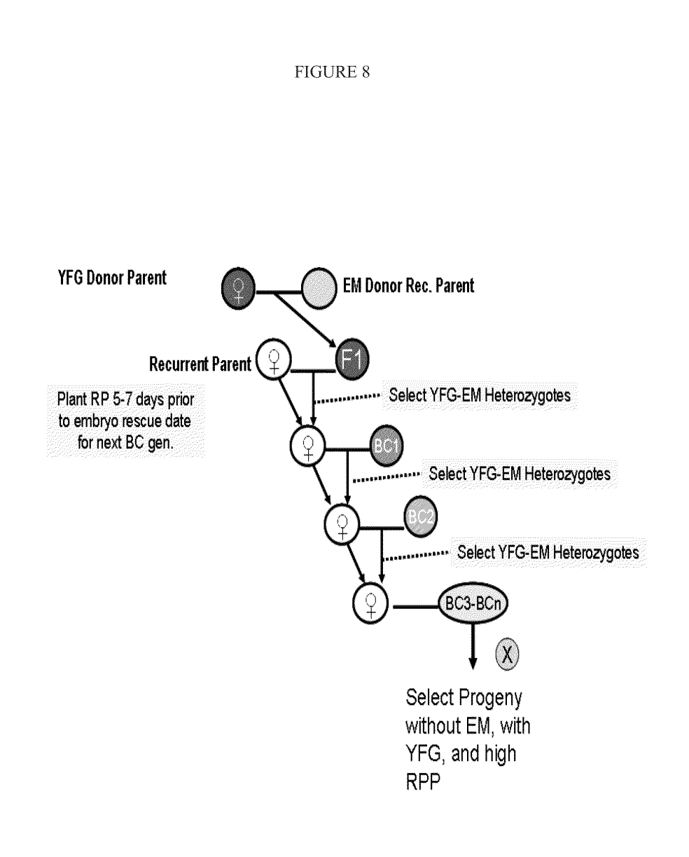

Another scheme is indicated in FIG. 8, where the desired gene is called “YFG.” As illustrated by FIG. 8, the Clearfield gene (for example) in the Clearfield donor is crossed to EM mutant parent, giving a heterozygous EM/CL 1. The F1 progeny (used as the donor for the CL trait) can be crossed to an elite recurrent parent. At each of 3 backcross stages, progeny of each cross is then crossed to the recurrent parent (with each backcross, selecting for EM/CL from EM, CL and EM/CL progeny) using molecular markers to recover the recurrent parent. By third backcross using molecular markers, one can recover most of the genome of the recurrent parent which will contain the gene of interest (the Clearfield gene).

The same can be accomplished after 5 rounds of backcrossing using visual selection (without molecular markers). However, molecular markers and the subject early gene greatly speed the cycle. For example, each cycle can be reduced by 20 days, for example. Thus, three to four generations, for example, can be Obtained per year by practice of the subject invention.

In summary, across can be made between the ‘Donor’ and ‘Recurrent’ parent. Then the F1 and subsequent generations are crossed (backcrossed) to the recurrent parent. The backcross generations converge on a single genotype. The genetic contribution of the ‘Donor’ parent will be halved each generation.

A satisfactory recurrent parent is usually from an established cultivar. A donor parent typically provides a desirable characteristic. There are a sufficient number of backcrosses to reconstitute the recurrent parent.

These backcrossing methods can provide the breeder a high degree of control. The traits to be improved can be described in advance. These methods are repeatable. Extensive field trials are not required. In addition, there is a reduced need for taking notes and record keeping.

5) Utility of the sunflower early flowering mutant gene offers exciting possibilities for known prior disclosure of transgenic development in other crops by broadening the adaptability of economically superior genetic combinations. There is no known prior disclosure of a similar dominant gene action occurring in other plant species. The gene can also be further mapped and sequenced. Gene optimizations can also be made for additional transformation. A TaqMan or an invader assay can also be developed to assist introgression.

### Example 9

### Additional Marker Development

### Materials and Methods

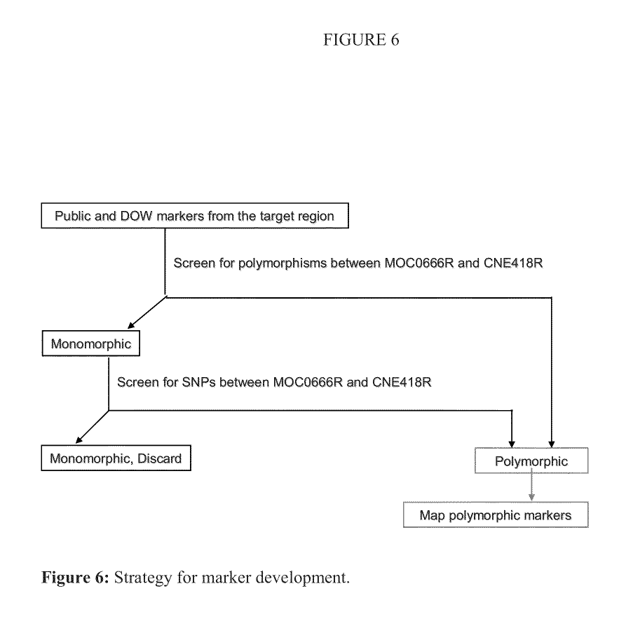

A strategy for marker development is summarized in this Example and is depicted in FIG. 6. Markers were selected and developed for the lower telomere region of linkage group 5 (LG 5) and were screened for polymorphisms between parental lines MOC0666R and CNE418R of the MOC0666R×CNE418R mapping population, which was previously used to map the early flowering (EF) mutant gene. Polymorphic markers were then mapped in the MOC0666R×CNE418R mapping population. For markers monomorphic between MOC0666R and CNE418R, primers were designed to amplify their genomic loci. Amplicons from both MOC0666R and CNE418R were cloned and sequenced to identify single nucleotide polymorphisms (SNPs), if any, between the two parental lines. TaqMan MGB Allelic Discrimination assays were developed to map identified SNPs. JoinMap 4.0 (Van Ooijen, 2004) was employed to map newly developed markers.

### Results

### SSR Marker Development

Here SSR markers were screened for polymorphisms between MOC0666R and CNE418R (Table 17). One SSR marker (HA1805) was polymorphic, and amplicons from MOC0666R and CNE418R were 240 bp and 235 bp, respectively. Correspondingly, the MOC0666R×CNE418R mapping population was genotyped with HA1805 using the following PCR primers and reaction conditions. PCR products were resolved on ABI 3730 Sequencer.

HA1805 Forward Primer:
(SEQ ID NO: 61)
5′-6FAM-GAAGTTGGGAGGGTTGTTCAAG-3′
HA1805 Reverse Primer:
(SEQ ID NO: 62)
5′- CCTCCTGTTGGAACACCAAAT-3′

PCR components:

4 ng gDNA

1X PCR buffer (Qiagen, Valencia, Calif.)

0.25 μM Forward primer

0.25 μM Reverse primer

1 mM MgCl₂

0.1 mM of each dNTP

0.4% PVP

0.04 Units HotStar Tag DNA polymerase (Qiagen, Valencia, Calif.)

Total Volume: 4.8 μl

Thermocycler setup:

Step 1: 94° C. for 12 minutes

Step 2: 94° C. for 30 seconds

Step 3: 55° C. for 30 seconds

Step 4: 72° C. for 30 seconds

Step 5: repeat steps 2, 3 and 4 for 35 cycles

Step 6: 72° C. for 30 minutes

### SNP Marker Development

Five pairs of primers were used to amplify five genomic loci from both MOC0666R and CNE418R to develop SNP markers (Table 17).

TABLE 17
Markers tested.

| F Name | Sequence | R Name | Sequence | Note |
|---|---|---|---|---|
| 1) SSR | | | | |
| HA1659F- | GGTCTTTTGTTTAGAGG | HA1659R | CGTTTCCCCATTTACATCA | |
| NED | CGTGAT | | TCTT | |
| | (SEQ ID NO: 68) | | (SEQ ID NO: 70) | |
| HA1805F- | GAAGTTGGGAGGGTTGT | HA1805R | CCTCCTGTTGGAACACCAA | polymorphic |
| FAM | TCAAG | | AT | |
| | (SEQ ID NO: 61) | | (SEQ ID NO: 62) | |
| ZVG24ssrF- | AAGCTTTGATCCGGGTT | ZVG24ssrR | GCCTTTCTTCCCAGCA | |
| FAM | TCT | | (SEQ ID NO: 71) | |
| | (SEQ ID NO: 69) | | | |
| 2) SNP | | | | |
| ZVG23snpF | CTGAATTCGAACACGAG | ZVG23snpR | TCTCCAGCCTTCAGCGTTA | monomorphic |
| | CAA | | T | |
| | (SEQ ID NO: 72) | | (SEQ ID NO: 77) | |
| ZVG24snpF | TGAGTCTTACGTGGCAA | ZVG24snpR | TGTCGCACAGGAAGTTTG | monomorphic |
| | ACG | | AG | |
| | (SEQ ID NO: 73) | | (SEQ ID NO: 78) | |
| HT120F | TACAAAGAAAGAGGGC | HT120R | AACATAAGAAAACCATAT | SNPs |
| | GAGA | | TCAAATCA | |
| | (SEQ ID NO: 74) | | (SEQ ID NO: 79) | |
| HT137F | TCCGTCTGGACTCAAAA | HT137R | CCAGAAGCACTTCAAGAG | SNPs |
| | CTC | | GA | |
| | (SEQ ID NO: 75) | | (SEQ ID NO: 80) | |
| HT151F | GTACGTCAACGATGCAT | HT151R | TATCATTCCTCCACCGAGA | monomorphic |
| | TTG | | A | |
| | (SEQ ID NO. 76) | | (SEQ ID NO: 81) | |

Two primer pairs (ZVG23snpF/R and ZVG24snpF/R) were designed based on sequences from restriction fragment length polymorphism (RFLP) probes ZVG23 and ZVG24 (Kolkman et al. 2007). Primer sequences for HT120F/R, HT137F/R, and HT151F/R were from Lai et al. (2005). SNPs were found in the amplicons from HT120F/R and HT137F/R. A TaqMan MGB Allelic Discrimination assay was developed for one SNP locus in the HT120F/R amplicon see below), and the MOC0666R×CNE418R mapping population was genotyped using this assay.

There were two SNP loci (underlined) in the HT120F/R amplicons from MOC0666R/CNE418R.

TACAAAGAAAGAGGGCGAGAATTGCGGATAAAAAGAAAAGAATTGCGAAG
GCGAAATCCGAGGCY(T/C)GCAGAGTATCAGAAACTTCTTGCTACGAGA
TTGAAGGAACAGAGAGAAAGGCGGAGCGAR(A/G)AGCTTAGCAAAGAAA
AGGTCGAGACTTTCTGCTGCTTCGAAACCTTCTATTGCAGCATAAGTTAA
CAAGTTTTCAGGGTAATTTCACAATGATTTGAATATGGTTTTCTTATGTT
(SEQ ID NO: 63
(wild-type, where Y = T and R = A) and
SEQ ID NO: 82
(early mutant, where Y = C and R = G))

The TaqMan Assay was developed for the R-locus, and the SNPO marker was designated DAS HA SNP 2008. The following sequences were used as indicated:

(SEQ ID NO: 64)
5′-ACGAGATTGAAGGAACAGAGAGAAA-3′
(Forward Primer,
(SEQ ID NO: 65)
5′-GCAGCAGAAAGTCTCGACCTTT-3′
(Reverse Primer,
(Probe 1; SEQ ID NO: 66)
5′-6FAM-CGGAGCGAGAGCT-3′,
and
(Probe 2; SEQ ID NO: 67)
5′-VIC-AGCGAAAGCTTAGC-3′.

Real-Time PCR components:

25 ng gDNA

1X Taqman Universal PCR Master Mix

22.5 μM Forward Primer

22.5 μM Reverse Primer

5 μM Probe 1

5 μM Probe 2

Total Volume: 25 μl

Bio-Rad iCycler setup:

Step 1: 95° C. for 15 minutes

Step 2: 94° C. for 30 seconds

Step 3: 60° C. for 1 minute

Step 4: repeat steps 2 and 3 for 65 cycles

Step 5: 4° C. forever

### Mapping New Markers

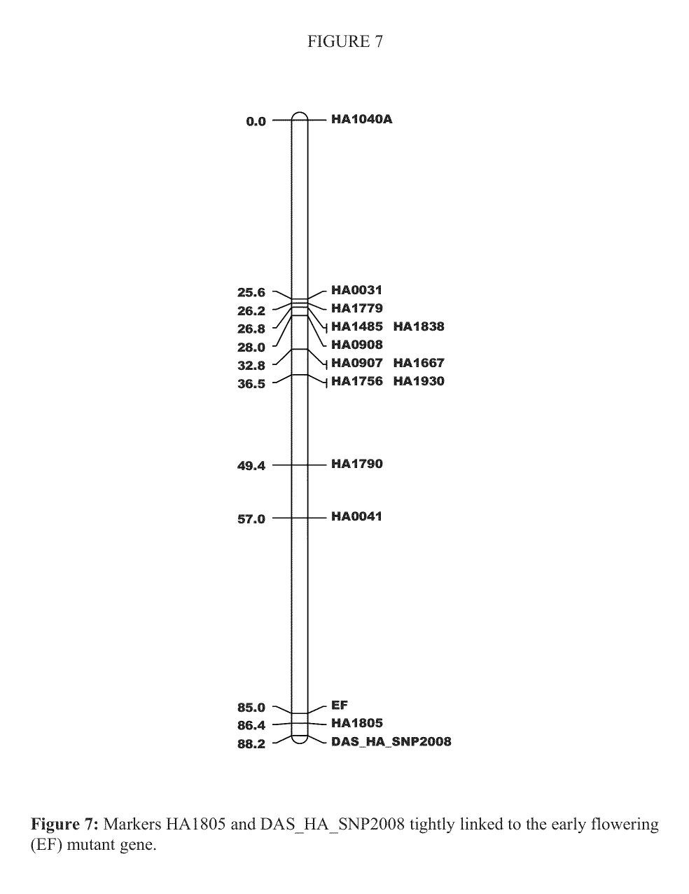

JoinMap 4.0 (Van Ooijen, 2006) was used to map HA1805 and DAS HA SNP 2008 (FIG. 7). Both HA1805 and DAS HA SNP 2008 were tightly linked to the EF mutant gene, 1.4 and 1.8 cM below the EF mutant gene, respectively. Both markers are good-quality, co-dominant markers that can be readily used to, for example, facilitate the selection for early flowering in breeding programs.

### References for Example 9

Kolkman, J. M., S. T. Berry, A. J. Leon, M. B. Slabaugh, S. Tang, W. Gao, D. K. Shintani, J. M. Burke, and S. J. Knapp. 2007. Single nucleotide polymorphisms and linkage disequilibrium in sunflower. Genetics 177: 457-468.

Lai, Z., K. Livingstone, Y. Zou, S. A. Church, S. J. Knapp, J. Andrews, and L. H. Rieseberg. 2005. Theor Appl Genet 111: 1532-1544.

Van Ooijen, J. W. 2006. JoinMap 4.0, Software for the calculation of genetic linkage maps in experimental populations. Kyazma B. V., Wageningen, Netherlands.

1. A method of determining if a genomic test sample comprises a gene capable of conferring an early flowering and/or early maturity phenotype on a plant, said genomic test sample being obtained from a test plant or tissue, seed, or a part of said test plant, said test plant comprising a genome, and said method comprising assaying said test sample for presence of an early maturity gene as present in ATCC #PTA-8715.

2. The method of claim 1, wherein said method comprises assaying for presence of at least one single nucleotide polymorphism (SNP) in said genome, wherein presence of said at least one SNP indicates presence of said early flowering gene in said genome, and absence of said at least one SNP in said genome indicates a wild-type plant lacking said gene.

3. The method of claim 2, said method comprising obtaining genomic DNA from said test plant, amplifying a segment of said genomic DNA to form an amplicon, and determining if said amplicon comprises said at least one SNP.

4. The method of 3, said method comprising sequencing said amplicon.

5. The method of claim 2, wherein said SNP is in SEQ ID NO: 63 or SEQ ID NO: 82

6. A method of producing an early maturing plant, said method comprising the steps of claim 1, selecting an early maturing plant comprising said gene, and growing and propagating said early maturing plant.

7. A method of selective breeding, said method comprising the steps of claim 1, further comprising selecting a plant that tests positive for said gene, and further breeding said positive plant.

8. The method of claim 7, said method comprises crossing said positive plant with another plant.

9. The method of claim 7 wherein said plant is a sunflower.

10. The method of claim 8 wherein said plant is crossed with a sunflower plant of a line selected from the group consisting of an ornamental line and a confectionary line.

11. A method of propagating a plant, said plant having an early flowering phenotype, said method comprises the steps of claim 1, said method further comprising growing said positive plant, and self-crossing said positive plant.

12. A method of selecting for an early maturing plant, said method comprising the steps of claim 1, and selecting said early maturing plant for further breeding and/or propagation.

13. A sunflower plant identified according to claim 1.

14. A part of a plant of claim 13, said part comprising said gene.

15. The plant part of claim 14, wherein said part is a seed or pollen.

16. A method of determining if a test plant comprises an early flowering gene, said test plant comprising a genome, and said method comprising assaying said plant for presence of an early maturity gene as present in ATCC #PTA-8715.

17. The method of claim 16, wherein presence of SEQ ID NO:66 in said genome indicates presence of said early flowering gene in said genome, and presence of SEQ ID NO:67 in said genome indicates a wild-type plant.

18. The early maturing sunflower plant of claim 13 comprising a mutated, dominant single gene that causes early flowering and/or maturation, wherein said gene is located at map position 85 cM on the lower telomere region of linkage group 5.

19. The plant of claim 18, wherein said gene can be mapped via DNA markers, microsatellite or SSR markers to the lower telomere region of linkage group 5.

20. The early maturing sunflower plant of claim 13 comprising a mutated, dominant single gene that causes early flowering and/or maturation, wherein said gene is associated with marker HA1805, amplifiable with primers comprising SEQ ID NO:61 and SEQ ID NO:62, and with marker DAS HA SNP 2008, amplifiable with primers comprising SEQ ID NO:64 and SEQ ID NO:65, and where said gene is in the lower telomere region of linkage group 5.


SEQUENCE LISTING (82 sequences)

SEQ ID NO: 1 - DNA, 22 bp, Helianthus annuus
aaatccacaaggatgctcaatc

SEQ ID NO: 2 - DNA, 24 bp, Helianthus annuus
ggagatcatacaaagcgttatcgt

SEQ ID NO: 3 - DNA, 23 bp, Helianthus annuus
tttcgtgatggtgattgatgatt

SEQ ID NO: 4 - DNA, 23 bp, Helianthus annuus
cagcaactctgaccgtttcatta

SEQ ID NO: 5 - DNA, 21 bp, Helianthus annuus
cattgaggacgagaagccagt

SEQ ID NO: 6 - DNA, 22 bp, Helianthus annuus
gttccgtaccctgtttgagctt

SEQ ID NO: 7 - DNA, 20 bp, Helianthus annuus
tgttcacagctcccgtctaa

SEQ ID NO: 8 - DNA, 21 bp, Helianthus annuus
cacacacacaacaacctgacc

SEQ ID NO: 9 - DNA, 20 bp, Helianthus annuus
gccgtgaataatgggattga

SEQ ID NO: 10 - DNA, 20 bp, Helianthus annuus
gattgggtcagcttgtgtga

SEQ ID NO: 11 - DNA, 21 bp, Helianthus annuus
ccctggagtgtatgtccgtta

SEQ ID NO: 12 - DNA, 20 bp, Helianthus annuus
atccgtctgctgcctaatcc

SEQ ID NO: 13 - DNA, 22 bp, Helianthus annuus
tgaaacgtagtaacctgccaaa

SEQ ID NO: 14 - DNA, 20 bp, Helianthus annuus
ttggacgacctcggtatctt

SEQ ID NO: 15 - DNA, 23 bp, Helianthus annuus
tttcgtgatggtgattgatgatt

SEQ ID NO: 16 - DNA, 23 bp, Helianthus annuus
cagcaactctgaccgtttcatta

SEQ ID NO: 17 - DNA, 23 bp, Helianthus annuus
cttattccaaggacgcatagtcg

SEQ ID NO: 18 - DNA, 23 bp, Helianthus annuus
cgatggtatgattctcgacgtta

SEQ ID NO: 19 - DNA, 21 bp, Helianthus annuus
atttcaccctcactcccacac

SEQ ID NO: 20 - DNA, 21 bp, Helianthus annuus
taccggctggatatggagaat

SEQ ID NO: 21 - DNA, 23 bp, Helianthus annuus
gggaagtgggcttgtctatgtat

SEQ ID NO: 22 - DNA, 23 bp, Helianthus annuus
aacacaccgaaatcacctatgaa

SEQ ID NO: 23 - DNA, 22 bp, Helianthus annuus
agaggaatgagatcgggttgat

SEQ ID NO: 24 - DNA, 21 bp, Helianthus annuus
gtgggacaactcagcaacgtc

SEQ ID NO: 25 - DNA, 24 bp, Helianthus annuus
gaacatggccataactcatagacg

SEQ ID NO: 26 - DNA, 16 bp, Helianthus annuus
ccttcgacccaacatc

SEQ ID NO: 27 - DNA, 21 bp, Helianthus annuus
acgcacatgagagagaaagag

SEQ ID NO: 28 - DNA, 20 bp, Helianthus annuus
accttcgacccaacatcaag

SEQ ID NO: 29 - DNA, 22 bp, Helianthus annuus
atttgttcctggttcggtatcc

SEQ ID NO: 30 - DNA, 22 bp, Helianthus annuus
catgtctgatcttcgggacttc

SEQ ID NO: 31 - DNA, 21 bp, Helianthus annuus
ctcacgaaactcttcatgctg

SEQ ID NO: 32 - DNA, 18 bp, Helianthus annuus
ctctcacacttactgaac

SEQ ID NO: 33 - DNA, 23 bp, Helianthus annuus
cctaaggggatgaattctctttc

SEQ ID NO: 34 - DNA, 23 bp, Helianthus annuus
aacttccaatgttctccaaccat

SEQ ID NO: 35 - DNA, 20 bp, Helianthus annuus
ttgtcttcatctgcgtgtga

SEQ ID NO: 36 - DNA, 20 bp, Helianthus annuus
ttgctgttgttgatcggtgt

SEQ ID NO: 37 - DNA, 20 bp, Helianthus annuus
gtgcgttggctcttatggat

SEQ ID NO: 38 - DNA, 20 bp, Helianthus annuus
agtgatggcattcccaattt

SEQ ID NO: 39 - DNA, 22 bp, Helianthus annuus
gtgatccgagttgtggatgttc

SEQ ID NO: 40 - DNA, 22 bp, Helianthus annuus
gttagatggcaacccaagtgat

SEQ ID NO: 41 - DNA, 22 bp, Helianthus annuus
ggtgatgatggaggagcaactg

SEQ ID NO: 42 - DNA, 22 bp, Helianthus annuus
cactcaaccattgttctcccac

SEQ ID NO: 43 - DNA, 22 bp, Helianthus annuus
gaactccggttagtcttccgac

SEQ ID NO: 44 - DNA, 24 bp, Helianthus annuus
gcaattaagtctgcgtttcagttt

SEQ ID NO: 45 - DNA, 20 bp, Helianthus annuus
catgaacatcgccaattcag

SEQ ID NO: 46 - DNA, 20 bp, Helianthus annuus
tgcaaggaaccatcagaatc

SEQ ID NO: 47 - DNA, 20 bp, Helianthus annuus
ttgtcatgtatgggctttgg

SEQ ID NO: 48 - DNA, 20 bp, Helianthus annuus
atccaacaggtgtgcggaat

SEQ ID NO: 49 - DNA, 21 bp, Helianthus annuus
cacttcatcctctccctcctt

SEQ ID NO: 50 - DNA, 20 bp, Helianthus annuus
ggcgtgtgtgttgggttatt

SEQ ID NO: 51 - DNA, 21 bp, Helianthus annuus
acacgagtcccaacctgaatg

SEQ ID NO: 52 - DNA, 24 bp, Helianthus annuus
acctgaaatgcaaatctctacagg

SEQ ID NO: 53 - DNA, 22 bp, Helianthus annuus
taggcaataactttgggcgaat

SEQ ID NO: 54 - DNA, 23 bp, Helianthus annuus
cctgaaatgcaaatctctacagg

SEQ ID NO: 55 - DNA, 21 bp, Helianthus annuus
tccccaaacttgcgtgtaggt

SEQ ID NO: 56 - DNA, 23 bp, Helianthus annuus
cattacaaaccacagctccttcc

SEQ ID NO: 57 - DNA, 20 bp, Helianthus annuus
cctggaactgaaccgagaac

SEQ ID NO: 58 - DNA, 20 bp, Helianthus annuus
gccgtgaaacagagagagga

SEQ ID NO: 59 - DNA, 19 bp, Helianthus annuus
ctagcaaccaacctcattg

SEQ ID NO: 60 - DNA, 20 bp, Helianthus annuus
gtctccttctctttctcggc

SEQ ID NO: 61 - DNA, 22 bp, Helianthus annuus
gaagttgggagggttgttcaag

SEQ ID NO: 62 - DNA, 21 bp, Helianthus annuus
cctcctgttggaacaccaaat

SEQ ID NO: 63 - DNA, 240 bp, Helianthus annuus
tacaaagaaagagggcgagaattgcggataaaaagaaaagaattgcgaaggcgaaatccgaggctgcagagtatcagaaacttcttgctacgagattgaaggaacagagagaaaggcggagcgaaagcttagcaaagaaaaggtcgagactttctgctgcttcgaaaccttctattgcagcataagttaacaagttttcagggtaatttcacaatgatttgaatatggttttcttatgtt

SEQ ID NO: 64 - DNA, 25 bp, Helianthus annuus
acgagattgaaggaacagagagaaa

SEQ ID NO: 65 - DNA, 22 bp, Helianthus annuus
gcagcagaaagtctcgaccttt

SEQ ID NO: 66 - DNA, 13 bp, Helianthus annuus
cggagcgagagct

SEQ ID NO: 67 - DNA, 14 bp, Helianthus annuus
agcgaaagcttagc

SEQ ID NO: 68 - DNA, 23 bp, Helianthus annuus
ggtcttttgtttagaggcgtgat

SEQ ID NO: 69 - DNA, 20 bp, Helianthus annuus
aagctttgatccgggtttct

SEQ ID NO: 70 - DNA, 23 bp, Helianthus annuus
cgtttccccatttacatcatctt

SEQ ID NO: 71 - DNA, 16 bp, Helianthus annuus
gcctttcttcccagca

SEQ ID NO: 72 - DNA, 20 bp, Helianthus annuus
ctgaattcgaacacgagcaa

SEQ ID NO: 73 - DNA, 20 bp, Helianthus annuus
tgagtcttacgtggcaaacg

SEQ ID NO: 74 - DNA, 20 bp, Helianthus annuus
tacaaagaaagagggcgaga

SEQ ID NO: 75 - DNA, 20 bp, Helianthus annuus
tccgtctggactcaaaactc

SEQ ID NO: 76 - DNA, 20 bp, Helianthus annuus
gtacgtcaacgatgcatttg

SEQ ID NO: 77 - DNA, 20 bp, Helianthus annuus
tctccagccttcagcgttat

SEQ ID NO: 78 - DNA, 20 bp, Helianthus annuus
tgtcgcacaggaagtttgag

SEQ ID NO: 79 - DNA, 26 bp, Helianthus annuus
aacataagaaaaccatattcaaatca

SEQ ID NO: 80 - DNA, 20 bp, Helianthus annuus
ccagaagcacttcaagagga

SEQ ID NO: 81 - DNA, 20 bp, Helianthus annuus
tatcattcctccaccgagaa

SEQ ID NO: 82 - DNA, 240 bp, Helianthus annuus
tacaaagaaagagggcgagaattgcggataaaaagaaaagaattgcgaaggcgaaatccgaggccgcagagtatcagaaacttcttgctacgagattgaaggaacagagagaaaggcggagcgagagcttagcaaagaaaaggtcgagactttctgctgcttcgaaaccttctattgcagcataagttaacaagttttcagggtaatttcacaatgatttgaatatggttttcttatgtt